(19) United States
(12) Patent Application Publication
(10) Pub. No.: US 2022/0304917 A1
(43) Pub. Date: Sep. 29, 2022

(54) Personal Care Compositions and Methods

(71) Applicant: Colgate-Palmolive Company, New York, NY (US)

(72) Inventors: Edgar H Hernandez, Residencial Acueducto de Guadalupe (MX); Sharon KENNEDY, Randallstown, MD (US); Qiang WU, Hillsborough, NJ (US); Srdjan MAKSIMOVIC, Bridgewater, NJ (US); André MORGAN, Robbinsville, NJ (US); Kathy POTECHIN, Short Hills, NJ (US); Thomas BOYD, Metuchen, NJ (US)

(73) Assignee: Colgate-Palmolive Company, New York, NY (US)

(21) Appl. No.: 17/250,387

(22) PCT Filed: Oct. 5, 2020
(86) PCT No.: PCT/US2020/070614

§ 371 Date: Jan. 14, 2021

Related U.S. Application Data
(60) Provisional application No. 62/911,634, filed on Oct. 7, 2019.

(51) Int. Cl. A61K 8/9789; A61K 8/92; A61Q 15/00; A61K 8/60; A61K 8/25; A61K 8/34

(52) U.S. Cl. CPC A61K 8/9789; A61K 8/922; A61Q 15/00; A61K 8/60; A61K 8/25; A61K 8/34

(57) ABSTRACT

Personal care compositions containing antiperspirant active ingredients and a cannabinoid source present in an amount to achieve an anti-irritant effect on the skin. Methods of preparing the personal care composition and uses of the personal care composition are also disclosed.

### BACKGROUND

Underarm deodorants control odor by eliminating the bacteria that cause odor. Conventional antiperspirant salts, such as aluminum, aluminum, and/or zirconium salts, tend to be acidic in aqueous solution, a property which makes them effective bactericides, thereby providing a deodorant benefit, but which can also cause skin irritation. In addition, long-acting antiperspirant compositions typically contain increased amounts of active ingredients as a route to obtaining sustained effectiveness. At the same time, skin sensitivity and vulnerability to various compounds may limit the practical upper concentration in personal care formulations. It is now believed that up to 50% of the population has sensitive skin with a reduced irritation threshold.

In recent years, medicinal and therapeutic uses of cannabinoids have garnered increased attention in both the media and within the scientific community. In the United States, cannabis laws have become steadily more liberal, with many states permitting the use of cannabinoids for medical purposes or for general recreational use. As public support grows, the numbers of these states are likely to increase and therefore support the efforts to clarify the potential therapeutic benefits of medical cannabis on various health outcomes.

Cannabidiol (CBD) is a naturally occurring cannabinoid in the Cannabis sativa plant, also known as marijuana. Cannabinoids are a class of diverse chemical compounds that act on cannabinoid receptors in cells that alter neurotransmitter release in the brain. There are at least 113 different cannabinoids isolated from Cannabis, exhibiting varied effects. While delta-9-tetrahydrocannabinol (THC) is the major active ingredient of Cannabis extracts, cannabidiol makes up about 40% of Cannabis extracts and has been studied for many different uses. It is known that cannabidiol lacks the psychoactive effects seen in many of the other cannabinoids including delta-9-tetrahydrocannabinol (THC). Cannabidiol has been speculated to have potential as a treatment for a wide range of medical conditions including arthritis, diabetes, alcohol use disorders, multiple sclerosis, chronic pain, schizophrenia, post-traumatic stress disorder (PTSD), depression, rare white matter disorders, antibiotic-resistant infections, epilepsy, inflammation, and other neurological disorders. CBD has also generally been found to possess potent antibacterial properties, anxiolytic, and anti-inflammatory properties.

Without being bound by theory, it is believed that cannabinoids such as CBD, with its antibacterial and anti-inflammatory properties, are well-suited to mitigate skin irritation, such as that resulting from application of a deodorant or an antiperspirant.

It is therefore desirable to develop and formulate efficacious anti-irritant ingredients into personal care products to mitigate potential irritant-induced redness, tingling, itching, or burning of the skin to a tolerable level for improved consumer compliance.

### BRIEF SUMMARY

The present inventors have discovered that the presence of a cannabinoid provides a surprising reduction of the irritation and inflammation which can be caused by active ingredients commonly used in personal care products, such as antiperspirant compositions.

The problem of sensitive skin affects a growing number of adults and children. It is now assumed that up to 50% of the world population have sensitive skin (L. Misery et al., Ann. Dermatol. Venereol. 2005, 132, 425-429). Sensitive skin describes a skin having a reduced irritation threshold for irritants, such as hyper-reactive, intolerant and also atopic skin. In the case of humans with sensitive, delicate or easily injured skin, a phenomenon called “stinging” can be observed. Typical adverse phenomena associated with the terms “stinging” or “sensitive skin” are reddening of the skin, tingling, prickling, tautness and burning of the skin and itching. They can be caused by stimulating environmental conditions, such as e.g. massage, action of surfactants, influence of weather, such as heat, cold, dryness and also damp heat, thermal radiation and UV radiation, e.g. from the sun, or psychological stress.

Therefore, in one embodiment, a personal care composition is provided for application to the skin or hair comprising a cosmetically acceptable carrier and an active ingredient (e.g., an antiperspirant active ingredient or a deodorant active ingredient) in combination with a cannabinoid (e.g. cannabidiol).

In another embodiment, a method of mitigating or reducing skin irritation provided by applying a composition comprising a cosmetically acceptable carrier and an antiperspirant active ingredient in combination with a cannabinoid (e.g. cannabidiol) to the skin or hair.

In further embodiment, the use of antiperspirant active and a cannabinoid (e.g. cannabidiol) to kill bacteria, reduce perspiration, and/or reduce body odor.

The invention also encompasses other personal care compositions for application to the skin, for example hand soaps or body washes, comprising a potentially irritating active ingredient and/or precursors thereof. The invention further provides methods of reducing sweat comprising applying the composition to skin, and methods of killing bacteria comprising contacting the bacteria with the composition.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

The invention therefore provides a personal care composition [Composition 1] for application to the skin or hair comprising a cosmetically acceptable carrier and about 0.001 to about 5.0 wt. % of a cannabinoid source, based on the total weight of the composition.
1.1 Composition of 1.0, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
1.2 Composition of 1.0 or 1.1, wherein the cannabinoid source comprises is a non-psychoactive cannabinoid.
1.3 Any of the preceding compositions, wherein the cannabinoid source comprises less than 0.3 wt. % Δ9-tetrahydrocannabinol (THC) relative to the total weight of the composition.
1.4 Any of the preceding compositions, wherein the cannabinoid source comprises less than 0.1 wt. % Δ9-tetrahydrocannabinol (THC) relative to the total weight of the composition.
1.5 Any of the preceding compositions, wherein the cannabinoid source comprises less than 0.01 wt. % Δ9-tetrahydrocannabinol (THC) relative to the total weight of the composition.
1.6 Any of the preceding compositions, wherein the cannabinoid source is substantially free of Δ9-tetrahydrocannabinol (THC).
1.7 Any of the preceding compositions, wherein the cannabinoid source comprises or consists of a cannabinoid selected from cannabichromene (CBC), cannabigerol (CBG), cannabidiol (CBD), and cannabinol (CBN), and combinations thereof.
1.8 Any of the preceding compositions, wherein the cannabinoid source comprises or consists of:

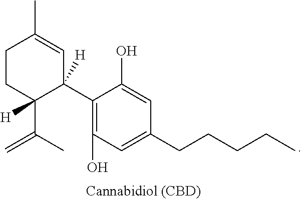 [SMILES: C=C(C)[C@@H]1CCC(C)=C[C@H]1c1c(O)cc(CCCCC)cc1O]

1.9 Any of the preceding compositions, wherein the cannabinoid source comprises hemp seed oil (HSO) or cannabis sativa seed oil (CSO), and wherein the HSO or CSO is a carrier for one or more cannabinoids (e.g., from 0.1%-7.5% by wt. of HSO or CSO, relative to the total weight of the composition) (e.g., about 5% CSO, by weight of the total composition).
1.10 Any of the preceding compositions, wherein the cannabinoid source comprises hemp seed oil and is a carrier for one or more cannabinoid.
1.11 Any of the preceding compositions, wherein the cannabinoid source comprises cannabis sativa seed oil and is a carrier for one or more cannabinoid.
1.12 The preceding composition, wherein the one or more cannabinoid is selected from: cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
1.13 The preceding composition, wherein the cannabinoid source comprises of a cannabinoid selected from cannabichromene (CBC), cannabigerol (CBG), cannabidiol (CBD), cannabinol (CBN), and combinations thereof.
1.14 The preceding composition, wherein cannabichromene (CBC), cannabigerol (CBG), cannabidiol (CBD), and/or cannabinol (CBN) are present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
1.15 Any of the preceding compositions, wherein the cannabinoid source comprises cannabidiol (CBD).
1.16 Any of the preceding compositions, comprising cannabidiol in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
1.17 Any of the foregoing compositions further comprising a metal-containing antiperspirant active ingredient.
1.18 The preceding composition, wherein the metal-containing antiperspirant active ingredient contains aluminum, magnesium, strontium, zirconium, zinc or a combination thereof.
1.19 Any of the compositions 1.17-1.18, wherein the metal-containing antiperspirant active ingredient is present in an amount of 1 to 40% by weight of the composition, optionally from 6, 7, 8, 9, 10, 11, 12, 13, or 14% up to 40% by weight of the composition, or, optionally, 10 to 30%, 11 to 25%, 12 to 20%, 13 to 15%, 14 to 20%, 15 to 20%, 11 to 15%, or 12 to 14% by weight of the composition.
1.20 Any of the foregoing compositions in a cosmetically acceptable base suitable for application to the skin, e.g., a cosmetically acceptable base comprising one or more of water-soluble alcohols (such as C₂₋₈ alcohols including ethanol); glycols (including propylene glycol, dipropylene glycol, tripropylene glycol and mixtures thereof); glycerides (including mono-, di- and triglycerides); medium to long chain organic acids, alcohols and esters; surfactants (including emulsifying and dispersing agents); additional amino acids; structurants (including thickeners and gelling agents, for example polymers, silicates and silicon dioxide); emollients; fragrances; and colorants (including dyes and pigments).
1.21 Any of the foregoing compositions, further comprising a soothing agent.
1.22 The preceding composition, wherein the soothing agent is selected from Aloe vera, allantoin, D-panthenol, turmeric, avocado oil and other vegetative oils, and lichen extract.
1.23 Any of the foregoing compositions, further comprising a fragrance component.
1.24 Any of the foregoing compositions, comprising water in an amount from about 10-75 wt. %, e.g. 20-60 wt. % based on total weight of the composition.
1.25 Any of the foregoing compositions, wherein the composition is substantially anhydrous, e.g., comprises less than 5% water.
1.26 Any of the foregoing compositions, wherein the composition is completely anhydrous, i.e., comprises 0% water.
1.27 Any of the foregoing compositions, wherein the composition is an oil-in-water (O/W) emulsion or a water-in-oil emulsion (W/O).
1.28 Any of the foregoing compositions, wherein the composition comprises an oil phase.
1.29 The preceding composition, wherein the oil phase comprises soybean oil, castor oil, palm kernel oil or combinations thereof.
1.30 Any of the foregoing compositions, wherein the composition is an antiperspirant and/or deodorant, e.g., an antiperspirant stick, an aerosol antiperspirant spray, or a liquid roll-on antiperspirant.
1.31 Any of the foregoing compositions, wherein the composition is a body wash, a shower gel, a bar soap, a shampoo, or hair conditioner.
1.32 Any of the foregoing compositions, for use to occlude pores.
1.33 Any of the foregoing compositions, for use to reduce sweat.

The invention further provides methods of reducing perspiration comprising applying an antiperspirant effective amount of any of Composition 1, et seq. to the skin, methods of reducing body odor comprising applying a deodorant-effective amount of any of Composition 1, et seq. to the skin, and methods of killing bacteria comprising contacting the bacteria with an effective antibacterial amount of a composition, e.g., any of Composition 1, et seq.

In another aspect, the invention provides a stick deodorant or antiperspirant composition [Composition 2] for application to the skin comprising a cosmetically acceptable carrier and about 0.001 to about 5.0 wt. % of a cannabinoid source, based on the total weight of the composition.
2.1 Composition 2, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
2.2 Any of the preceding compositions, wherein the cannabinoid source comprises is a non-psychoactive cannabinoid.
2.3 Any of the preceding compositions, wherein the cannabinoid source comprises less than 0.3 wt. % Δ9-tetrahydrocannabinol (THC) relative to the total weight of the composition.
2.4 Any of the preceding compositions, wherein the cannabinoid source comprises less than 0.1 wt. % Δ9-tetrahydrocannabinol (THC) relative to the total weight of the composition.
2.5 Any of the preceding compositions, wherein the cannabinoid source comprises less than 0.01 wt. % Δ9-tetrahydrocannabinol (THC) relative to the total weight of the composition.
2.6 Any of the preceding compositions, wherein the cannabinoid source is substantially free of Δ9-tetrahydrocannabinol (THC).
2.7 Any of the preceding compositions, wherein the cannabinoid source comprises or consists of a cannabinoid selected from cannabichromene (CBC), cannabigerol (CBG), cannabidiol (CBD), and cannabinol (CBN), and combinations thereof.
2.8 Any of the preceding compositions, wherein the cannabinoid source comprises or consists of:

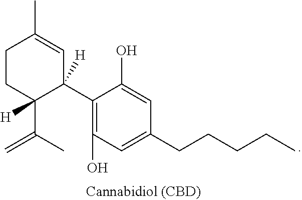 [SMILES: C=C(C)[C@@H]1CCC(C)=C[C@H]1c1c(O)cc(CCCCC)cc1O]

2.9 Any of the preceding compositions, wherein the cannabinoid source comprises hemp seed oil (HSO) or cannabis sativa seed oil (CSO), and wherein the HSO or CSO is a carrier for one or more cannabinoids. (e.g., from 0.1%-7.5% by wt. of HSO or CSO, relative to the total weight of the composition) (e.g., about 5% CSO, by weight of the total composition)
2.10 Any of the preceding compositions, wherein the cannabinoid source comprises hemp seed oil and is a carrier for one or more cannabinoid.
2.11 Any of the preceding compositions, wherein the cannabinoid source comprises cannabis sativa seed oil and is a carrier for one or more cannabinoid.
2.12 The preceding composition, wherein the one or more cannabinoid is selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
2.13 The preceding composition, wherein the cannabinoid source comprises a cannabinoid selected from cannabichromene (CBC), cannabigerol (CBG), cannabidiol (CBD), cannabinol (CBN), and combinations thereof.
2.14 The preceding composition, wherein cannabichromene (CBC), cannabigerol (CBG), cannabidiol (CBD), and/or cannabinol (CBN), are present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
2.15 The composition of 2.10, wherein the cannabinoid source comprises cannabidiol (CBD).
2.16 Any of the preceding compositions, comprising cannabidiol in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
2.17 Any of the preceding compositions in a cosmetically acceptable base suitable for application to the skin, e.g., a cosmetically acceptable base comprising one or more of water-soluble alcohols (such as C₂₋₈ alcohols including ethanol); glycols (including propylene glycol, dipropylene glycol, tripropylene glycol and mixtures thereof); glycerides (including mono-, di- and triglycerides); medium to long chain organic acids, alcohols and esters; surfactants (including emulsifying and dispersing agents); additional amino acids; structurants (including thickeners and gelling agents, for example polymers, silicates and silicon dioxide); emollients; fragrances; and colorants (including dyes and pigments).
2.18 Any of the preceding compositions, further comprising a metal-containing antiperspirant active ingredient contains aluminum, magnesium, strontium, zirconium, zinc or a combination thereof.
2.19 Any of the preceding compositions, further comprising activated charcoal.
2.20 The preceding composition, wherein the metal-containing antiperspirant active ingredient is present in an amount of 1 to 40% by weight of the composition, optionally from 6, 7, 8, 9, 10, 11, 12, 13, or 14% up to 40% by weight of the composition, or, optionally, 10 to 30%, 11 to 25%, 12 to 20%, 13 to 15%, 14 to 20%, 15 to 20%, 11 to 15%, or 12 to 14% by weight of the composition.
2.21 Any of the preceding compositions, further comprising a non-volatile emollient.
2.22 Any of the preceding compositions, further comprising an emollient selected from C₁₂₋₁₅ alkyl benzoate, PPG-14 butyl ether, PPG-3 myristyl ether, secondary alcohol ethoxylates, coconut oil, rice wax, shea butter cocoa butter, stearyl alcohol, stearic acid and salts thereof, glyceryl monoricinoleate, isobutyl palmitate, glyceryl monostearate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, tea tree oil, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide.
2.23 Any of the preceding compositions, further comprising an emollient selected from C₁₂₋₁₅ alkyl benzoate, PEG-8 distearate or sodium stearate.
2.24 Any of the preceding compositions, further comprising a volatile emollient.
2.25 Any of the preceding compositions, further comprising a volatile emollient selected from cyclomethicone.
2.26 Any of the foregoing compositions, wherein the composition comprises an oil phase.
2.27 The preceding composition, wherein the oil phase comprises soybean oil, castor oil, palm kernel oil or combinations thereof.
2.28 Any of the preceding compositions, further comprising an antioxidant selected from citric acid, butylated hydroxytoluene, pentaerythrityl tetra-di-t-butyl hydroxyhydrocinnamate.
2.29 The preceding composition, wherein the antioxidant is present in an amount of about 0.1 to about 1 wt. %.
2.30 Any of the preceding compositions, comprising:

| Ingredient | Wt. % |
|---|---|
| Activated Aluminum Zirconium Tetrahydroclorex Glycine | 8-18 |
| Palm Kernel Oil | 30-45 |
| Cyclomethicone | 5-15 |
| C₁₂₋₁₅ Alkyl Benzoate | 10-25 |
| PEG-8 Distearate | 2-6 |
| Soybean Oil | 2-6 |
| 50% Citric Acid Soln. | 0.01-0.1 |
| Pentaerythrityl tetra-di-t-butyl hydroxyhydrocinnamate | 0.001-0.1 |
| Butylated hydroxytoluene | 0.01-1 |
| Synthetic Wax | 5-15 |
| Cannabis Sativa Seed Oil (5% CBD) | 0.001-5 |

2.31 Any of the preceding compositions, comprising:

| Ingredient | Wt. % |
|---|---|
| Polypropylene Glycol | 55-75 |
| Sodium Stearate | 5-15 |
| Stearyl Alcohol | 0.01-1 |
| Water | 15-25 |
| EDTA 62% Soln. | 0.001-0.01 |
| Sodium Chloride | 0.1-1 |
| Colorants | 0.0001-0.001 |
| Fragrance | 1-3 |
| Cannabis Sativa Seed Oil (5% CBD) | 0.001-5 |

In another aspect, the invention provides a roll-on deodorant or antiperspirant composition [Composition 3] for application to the skin comprising a cosmetically acceptable carrier and about 0.001 to about 5.0 wt. % of a cannabinoid source, based on the total weight of the composition.
3.1 Composition 3, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
3.2 Any of the preceding compositions, wherein the cannabinoid source comprises is a non-psychoactive cannabinoid.
3.3 Any of the preceding compositions, wherein the cannabinoid source comprises less than 0.3 wt. % Δ9-tetrahydrocannabinol (THC) relative to the total weight of the composition.
3.4 Any of the preceding compositions, wherein the cannabinoid source comprises less than 0.1 wt. % Δ9-tetrahydrocannabinol (THC) relative to the total weight of the composition.
3.5 Any of the preceding compositions, wherein the cannabinoid source comprises less than 0.01 wt. % Δ9-tetrahydrocannabinol (THC) relative to the total weight of the composition.
3.6 Any of the preceding compositions, wherein the cannabinoid source is substantially free of Δ9-tetrahydrocannabinol (THC).
3.7 Any of the preceding compositions, wherein the cannabinoid source comprises or consists of a cannabinoid selected from cannabichromene (CBC), cannabigerol (CBG), cannabidiol (CBD), and cannabinol (CBN), and combinations thereof.
3.8 Any of the preceding compositions, wherein the cannabinoid source comprises or consists of:

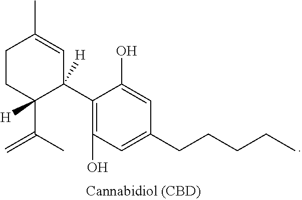 [SMILES: C=C(C)[C@@H]1CCC(C)=C[C@H]1c1c(O)cc(CCCCC)cc1O]

3.9 Any of the preceding compositions, wherein the cannabinoid source comprises hemp seed oil (HSO) or cannabis sativa seed oil (CSO), and wherein the HSO or CSO is a carrier for one or more cannabinoids (e.g., from 0.1%-7.5% by wt. of HSO or CSO, relative to the total weight of the composition) (e.g., about 5% CSO, by weight of the total composition).
3.10 Any of the preceding compositions, wherein the cannabinoid source comprises hemp seed oil and is a carrier for one or more cannabinoid.
3.11 Any of the preceding compositions, wherein the cannabinoid source comprises cannabis sativa seed oil and is a carrier for one or more cannabinoid.
3.12 The preceding composition, wherein the one or more cannabinoid is selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
3.13 The preceding composition, wherein the one or more cannabinoid is selected from cannabichromene (CBC), cannabigerol (CBG), cannabidiol (CBD), cannabinol (CBN), and combinations thereof.
3.14 The preceding composition, wherein cannabichromene (CBC), cannabigerol (CBG), cannabidiol (CBD), and/or cannabinol (CBN), are present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
3.15 Any of the preceding compositions, wherein the cannabinoid source comprises cannabidiol (CBD).
3.16 Any of the preceding compositions, wherein cannabidiol (CBD) is present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
3.17 Any of the preceding compositions in a cosmetically acceptable base suitable for application to the skin, e.g., a cosmetically acceptable base comprising one or more of water-soluble alcohols (such as C₂₋₈ alcohols including ethanol); glycols (including propylene glycol, dipropylene glycol, tripropylene glycol and mixtures thereof); glycerides (including mono-, di- and triglycerides); medium to long chain organic acids, alcohols and esters; surfactants (including emulsifying and dispersing agents); additional amino acids; structurants (including thickeners and gelling agents, for example polymers, silicates and silicon dioxide); emollients; fragrances; and colorants (including dyes and pigments).
3.18 Any of the preceding compositions comprising water in an amount of about 30-80 wt. %, about 40-70 wt. %, about 40-60 wt. %, about 50-70 wt. %, about 40 wt. %, about 45 wt. %, about 50 wt. %, about 55 wt. %, about 60 wt. %, about 65 wt. %, or about 70 wt. %.
3.19 Any of the preceding compositions, further comprising activated charcoal.
3.20 Any of the preceding compositions, further comprising polymeric thickeners selected from polyamides, cellulose derivatives (e.g., hydroxypropylcellulose, hydroxypropyl methyl cellulose, etc.) and natural or synthetic gums, such as polyglycerides including agar, agarose, pectin, or guars or mixtures or combinations thereof. One class of materials worthy of attention for thickening a water-immiscible phase comprises derivatives of hydrolysed starch or other polysaccharides, including in particular esterified dextrins, such as dextrin palmitate.
3.21 Any of the preceding composition, further comprising a cellulose derivative selected from hydroxypropylcellulose and hydroxypropyl methyl cellulose in an amount of about 0.5-1.5 wt. %.
3.22 Any of the preceding compositions, further comprising an emollient selected from C₁₂₋₁₅ alkyl benzoate, PPG-14 butyl ether, PPG-3 myristyl ether, secondary alcohol ethoxylates, coconut oil, rice wax, shea butter, cocoa butter, stearyl alcohol, stearic acid and salts thereof, glyceryl monoricinoleate, isobutyl palmitate, glyceryl monostearate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, tea tree oil, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide.
3.23 Any of the preceding compositions, further comprising one or more of propylene glycol in an amount of about 6-18 wt. %, a secondary alcohol ethoxylate (e.g., Tergitol) in an amount of about 1.5-2.5 wt. %, and/or stearyl alcohol (e.g., Steareth 20, Steareth 2, etc.) in an amount of about 0.5-1.5 wt. %.
3.24 Any of the foregoing compositions, wherein the composition comprises an oil phase.
3.25 The preceding composition, wherein the oil phase comprises soybean oil, castor oil, palm kernel oil or combinations thereof.
3.26 Any of the preceding compositions, further comprising a metal-containing antiperspirant active ingredient contains aluminum, magnesium, strontium, zirconium, zinc or a combination thereof.
3.27 The preceding composition, wherein the metal-containing antiperspirant active ingredient is present in an amount of 1 to 40% by weight of the composition, optionally from 6, 7, 8, 9, 10, 11, 12, 13, or 14% up to 40% by weight of the composition, or, optionally, 10 to 30%, 11 to 25%, 12 to 20%, 13 to 15%, 14 to 20%, 15 to 20%, 11 to 15%, or 12 to 14% by weight of the composition.
3.28 Any of the preceding compositions, further comprising an antioxidant selected from citric acid, butylated hydroxytoluene, pentaerythrityl tetra-di-t-butyl
3.29 Any of the preceding compositions, wherein the composition comprises:

| Description | wt. % |
|---|---|
| Water | 40-60 |
| Hydroxypropyl methylcellulose | 0.5-1.5 |
| Propylene Glycol | 5-15 |
| Polyethylene Glycol (PEG 600) | 1-3 |
| 50% Aluminum Chlorhydroxide Soln. | 20-40 |
| Tergitol 15-S-12 | 1.5-2.5 |
| Fragrance agents | 0.5-1.5 |
| Cannabis Sativa Seed Oil (5% CBD) | 0.001-5 |

3.30 Any of the preceding compositions, wherein the composition comprises:

| Description | wt. % |
|---|---|
| Water | 50-70 |
| Steareth-20 | 0.5-1.5 |
| Caprylyl Glycol | 0.01-0.5 |
| Stearyl Ether | 1-2 |
| Steareth-2 | 1.5-3 |
| Soybean oil | 2-4 |
| Butylated hydroxytoluene | 0.01-0.1 |
| EDTA 62% Soln. | 0.01-0.5 |
| 50% Aluminum Chlorhydroxide Soln. | 20-40 |
| Fragrance agents | 0.5-1.5 |
| Cannabis Sativa Seed Oil (5% CBD) | 0.001-5 |

In another aspect, the invention provides an aerosol deodorant or antiperspirant composition [Composition 4] for application to the skin comprising a cosmetically acceptable carrier and about 0.001 to about 5.0 wt. % of a cannabinoid source, based on the total weight of the composition.
4.1 Composition 4, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
4.2 Any of the preceding compositions, wherein the cannabinoid source comprises is a non-psychoactive cannabinoid.
4.3 Any of the preceding compositions, wherein the cannabinoid source comprises less than 0.3 wt. % Δ9-tetrahydrocannabinol (THC) relative to the total weight of the composition.
4.4 Any of the preceding compositions, wherein the cannabinoid source comprises less than 0.1 wt. % Δ9-tetrahydrocannabinol (THC) relative to the total weight of the composition.
4.5 Any of the preceding compositions, wherein the cannabinoid source comprises less than 0.01 wt. % Δ9-tetrahydrocannabinol (THC) relative to the total weight of the composition.
4.6 Any of the preceding compositions, wherein the cannabinoid source is substantially free of Δ9-tetrahydrocannabinol (THC).
4.7 Any of the preceding compositions, wherein the cannabinoid source comprises or consists of a cannabinoid selected from cannabichromene (CBC), cannabigerol (CBG), cannabidiol (CBD), and cannabinol (CBN), and combinations thereof.
4.8 Any of the preceding compositions, wherein the cannabinoid source comprises or consists of:

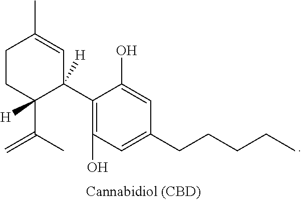 [SMILES: C=C(C)[C@@H]1CCC(C)=C[C@H]1c1c(O)cc(CCCCC)cc1O]

4.9 Any of the preceding compositions, wherein the cannabinoid source comprises hemp seed oil (HSO) or cannabis sativa seed oil (CSO), and wherein the HSO or CSO is a carrier for one or more cannabinoids (e.g., from 0.1%-7.5% by wt. of HSO or CSO, relative to the total weight of the composition) (e.g., about 5% CSO, by weight of the total composition).
4.10 Any of the preceding compositions, wherein the cannabinoid source comprises hemp seed oil and is a carrier for one or more cannabinoid.
4.11 Any of the preceding compositions, wherein the cannabinoid source comprises cannabis sativa seed oil and is a carrier for one or more cannabinoid.
4.12 The preceding composition, wherein the one or more cannabinoid is selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
4.13 The preceding composition, wherein the one or more cannabinoid is selected from cannabichromene (CBC), cannabigerol (CBG), cannabidiol (CBD), cannabinol (CBN), and combinations thereof.
4.14 The preceding composition, wherein cannabichromene (CBC), cannabigerol (CBG), cannabidiol (CBD), and/or cannabinol (CBN), are present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
4.15 Any of the preceding compositions, wherein the cannabinoid source comprises cannabidiol (CBD).
4.16 Any of the preceding compositions, wherein cannabidiol (CBD) is present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
4.17 Any of the preceding compositions in a cosmetically acceptable base suitable for application to the skin, e.g., a cosmetically acceptable base comprising one or more of water-soluble alcohols (such as C₂₋₈ alcohols including ethanol); glycols (including propylene glycol, dipropylene glycol, tripropylene glycol and mixtures thereof); glycerides (including mono-, di- and triglycerides); medium to long chain organic acids, alcohols and esters; surfactants (including emulsifying and dispersing agents); additional amino acids; structurants (including thickeners and gelling agents, for example polymers, silicates and silicon dioxide); emollients; fragrances; and colorants (including dyes and pigments).
4.18 Any of the preceding compositions comprising water in an amount of about 30-80 wt. %, about 40-70 wt. %, about 40-60 wt. %, about 50-70 wt. %, about 40 wt. %, about 45 wt. %, about 50 wt. %, about 55 wt. %, about 60 wt. %, about 65 wt. %, or about 70 wt. %.
4.19 Any of the preceding compositions, further comprising activated charcoal.
4.20 Any of the preceding compositions, further comprising a non-volatile emollient.
4.21 Any of the preceding compositions, further comprising an emollient selected from C₁₂₋₁₅ alkyl benzoate, PPG-14 butyl ether, PPG-3 myristyl ether, secondary alcohol ethoxylates, coconut oil, rice wax, shea butter, cocoa butter, stearyl alcohol, stearic acid and salts thereof, glyceryl monoricinoleate, isobutyl palmitate, glyceryl monostearate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, tea tree oil, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide.
4.22 Any of the preceding compositions, further comprising an emollient selected from C₁₂₋₁₅ alkyl benzoate in an amount of 5-18 wt. %, isopropyl palmitate in an amount of about 15-25 wt. %, and/or isopropyl myristate in an amount of about 15-25 wt. %.
4.23 Any of the preceding compositions, wherein the composition comprises:

| Material Description | wt. % |
|---|---|
| Isopropyl Palmitate | 15-25 |
| Isopropyl myristate | 15-25 |
| C₁₂₋₁₅ Alkyl Benzoate | 5-18 |
| Soybean oil | 2-6 |
| Bentone 27V CG | 1.5-3 |
| Propylene Carbonate | 0.1-1 |
| Aluminum Chlorhydroxide Powder | 30-50 |
| Cannabis Sativa Seed Oil (5% CBD) | 0.001-5 |

4.24 Any of the preceding compositions, wherein the composition comprises:

| Material Description | wt. % |
|---|---|
| 94% Ethyl Alcohol Soln. | 90-98 |
| Fragrance | 1-2 |
| Farnesol | 0.01-0.2 |
| Cannabis Sativa Seed Oil (5% CBD) | 0.001-5 |

In another aspect, the invention provides a solid deodorant or antiperspirant composition [Composition 5] for application to the skin comprising a cosmetically acceptable carrier and about 0.001 to about 5.0 wt. % of a cannabinoid source, based on the total weight of the composition.
5.1 Composition 5, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
5.2 Any of the preceding compositions, wherein the cannabinoid source comprises is a non-psychoactive cannabinoid.
5.3 Any of the preceding compositions, wherein the cannabinoid source comprises less than 0.3 wt. % Δ9-tetrahydrocannabinol (THC) relative to the total weight of the composition.
5.4 Any of the preceding compositions, wherein the cannabinoid source comprises less than 0.1 wt. % Δ9-tetrahydrocannabinol (THC) relative to the total weight of the composition.
5.5 Any of the preceding compositions, wherein the cannabinoid source comprises less than 0.01 wt. % Δ9-tetrahydrocannabinol (THC) relative to the total weight of the composition.
5.6 Any of the preceding compositions, wherein the cannabinoid source is substantially free of Δ9-tetrahydrocannabinol (THC).
5.7 Any of the preceding compositions, wherein the cannabinoid source comprises or consists of a cannabinoid selected from cannabichromene (CBC), cannabigerol (CBG), cannabidiol (CBD), and cannabinol (CBN), and combinations thereof.
5.8 Any of the preceding compositions, wherein the cannabinoid source comprises or consists of:

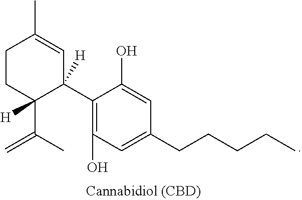 [SMILES: C=C(C)[C@@H]1CCC(C)=C[C@H]1c1c(O)cc(CCCCC)cc1O]

5.9 Any of the preceding compositions, wherein the cannabinoid source comprises hemp seed oil (HSO) or cannabis sativa seed oil (CSO), and wherein the HSO or CSO is a carrier for one or more cannabinoids (e.g., from 0.1%-7.5% by wt. of HSO or CSO, relative to the total weight of the composition) (e.g., about 5% CSO, by weight of the total composition).
5.10 Any of the preceding compositions, wherein the cannabinoid source comprises hemp seed oil and is a carrier for one or more cannabinoid.
5.11 Any of the preceding compositions, wherein the cannabinoid source comprises cannabis sativa seed oil and is a carrier for one or more cannabinoid.
5.12 The preceding composition, wherein the one or more cannabinoid is selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.
5.13 The preceding composition, wherein the one or more cannabinoid is selected from cannabichromene (CBC), cannabigerol (CBG), cannabidiol (CBD), cannabinol (CBN), and combinations thereof.
5.14 The preceding composition, wherein cannabichromene (CBC), cannabigerol (CBG), cannabidiol (CBD), and/or cannabinol (CBN), are present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
5.15 Any of the preceding compositions, wherein the cannabinoid source comprises cannabidiol (CBD).
5.16 Any of the preceding compositions, wherein cannabidiol (CBD) is present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
5.17 Any of the preceding compositions in a cosmetically acceptable base suitable for application to the skin, e.g., a cosmetically acceptable base comprising one or more of water-soluble alcohols (such as C₂₋₈ alcohols including ethanol); glycols (including propylene glycol, dipropylene glycol, tripropylene glycol and mixtures thereof); glycerides (including mono-, di- and triglycerides); medium to long chain organic acids, alcohols and esters; surfactants (including emulsifying and dispersing agents); additional amino acids; structurants (including thickeners and gelling agents, for example polymers, silicates and silicon dioxide); emollients; fragrances; and colorants (including dyes and pigments).
5.18 Any of the foregoing compositions, wherein the composition comprises an oil phase.
5.19 The preceding composition, wherein the oil phase comprises soybean oil, castor oil, palm kernel oil or combinations thereof.
5.20 Any of the preceding compositions, further comprising activated charcoal.
5.21 Any of the preceding compositions, further comprising a metal-containing antiperspirant active ingredient contains aluminum, magnesium, strontium, zirconium, zinc or a combination thereof.
5.22 The preceding composition, wherein the metal-containing antiperspirant active ingredient is present in an amount of 1 to 40% by weight of the composition, optionally from 6, 7, 8, 9, 10, 11, 12, 13, or 14% up to 40% by weight of the composition, or, optionally, 10 to 30%, 11 to 25%, 12 to 20%, 13 to 15%, 14 to 20%, 15 to 20%, 11 to 15%, or 12 to 14% by weight of the composition.
5.23 Any of the preceding compositions, further comprising an emollient selected from C₁₂₋₁₅ alkyl benzoate, PPG-14 butyl ether, PPG-3 myristyl ether, secondary alcohol ethoxylates, coconut oil, rice wax, shea butter, cocoa butter, stearyl alcohol, stearic acid and salts thereof, glyceryl monoricinoleate, isobutyl palmitate, glyceryl monostearate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, tea tree oil, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide.
5.24 Any of the preceding compositions, further comprising an emollient selected from dicaprylyl ether in an amount of 5-15 wt. %, stearyl alcohol in an amount of about 9-25 wt. % (e.g., 9-18 wt. % or 15-25 wt. %), and/or isopropyl myristate in an amount of about 15-25 wt. %.
5.25 Any of the preceding compositions, further comprising polymeric thickeners selected from polyamides, cellulose derivatives (e.g., hydroxypropylcellulose, hydroxypropyl methyl cellulose, etc.) and natural or synthetic gums, such as polyglycerides including agar, agarose, pectin, or guars or mixtures or combinations thereof. One class of materials worthy of attention for thickening a water-immiscible phase comprises derivatives of hydrolysed starch or other polysaccharides, including in particular esterified dextrins, such as dextrin palmitate.
5.26 Any of the preceding composition, further comprising maltodextrin in an amount of about 0.001-0.5 wt. %.
5.27 Any of the preceding compositions, wherein the composition comprises:

| Description | wt. % |
|---|---|
| Palm Kernel Oil | 30-40 |
| Dicaprylyl Ether | 5-15 |
| Soybean oil | 4-8 |
| Castor oil | 4-8 |
| Stearyl Alcohol | 9-18 |
| Aluminum Chlorhydroxide Powder | 25-35 |
| Olea Europaea Leaf Extract | 0.001-0.5 |
| Maltodextrin | 0.001-0.5 |
| Silica | 0.001-0.5 |
| Fragrance | 0.1-2 |
| Cannabis Sativa Seed Oil (5% CBD) | 0.001-5 |

5.28 Any of the preceding compositions, wherein the composition comprises:

| Description | wt. % |
|---|---|
| Palm Kernel Oil | 30-45 |
| Stearyl Alcohol | 15-25 |
| Caprylic/Capric Triglyceride | 5-10 |
| White Beeswax | 1-5 |
| Dicaprylyl Ether | 5-15 |
| Soybean oil | 6-10 |
| Castor oil | 6-10 |
| Zinc Oxide | 1-3 |
| Cannabis Sativa Seed Oil (5% CBD) | 0.001-5 |
| Fragrance | 1-2 |

5.29 Any of the preceding compositions, wherein the composition comprises:

| Description | wt. % |
|---|---|
| Propylene Glycol | 55-75 |
| Water | 20-30 |
| Sodium Stearate | 4-8 |
| Glyceryl Monolaurate | 0.1-2 |
| Ascorbic Acid | 0.01-0.1 |
| Aloe | 0.01-0.1 |
| Cannabis Sativa Seed Oil (5% CBD) | 0.001-5 |

A composition of any of Composition 1.0, et seq, Composition 2.0, et seq, Composition 3.0, et seq, Composition 4.0, et seq, and/or Composition 5.0, et seq, wherein the cannabinoid consists of cannabidiol (CBD), and CBD is the only cannabinoid present in the composition.

The invention further provides a method of making a composition comprising combining the antiperspirant active ingredient and a cannabinoid in a cosmetically acceptable base material.

As used herein, the term antiperspirant can refer to any material that can form a “plug” in a pore to reduce sweating, or antiperspirant refers to those materials classified as antiperspirants by the Food and Drug Administration under 21 CFR part 350. Antiperspirants may also be deodorants, particularly in the case of this invention, as the aluminum, magnesium, strontium, zirconium and zinc-containing active ingredients have antibacterial properties and can reduce odor-causing bacteria on the skin.

The antiperspirant active ingredients for use in the antiperspirant embodiments of the present invention include any compound, composition or other material having antiperspirant activity. Generally, any of the Category I active antiperspirant ingredients, listed in the Food and Drug Administration's Monograph on Antiperspirant Drug Products for overall-the-counter human use (Oct. 10, 1973) can be used. In addition, any new ingredient, not listed in the Monograph, can be incorporated as an antiperspirant active. Preferred antiperspirant actives include astringent metallic salts, especially inorganic and organic salts of aluminum, zirconium and zinc, as well as mixtures thereof. Particularly preferred are aluminum-containing and/or zirconium-containing salts or materials, such as aluminum halides, aluminum hydroxyhalides, zirconyl oxyhalides, zirconyl hydroxyhalides, and mixtures thereof. Especially useful antiperspirant actives suitable for use in the formulations include aluminum bromohydrate, aluminum chlorohydrate, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum zirconium chlorohydrate, aluminum zirconium trichlorohydrate, aluminum zirconium tetrachlorohydrate, aluminum zirconium pentachlorohydrate, aluminum zirconium octachlorohydrate, aluminum zirconium tetrachlorohydrex propylene glycol complex, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, aluminum chloride, aluminum sulfate, buffered aluminum sulfate, potassium alum, sodium aluminum chlorohydroxy lactate and combinations thereof.

The composition can be any type of personal care composition. In certain embodiments, the composition is any composition in which it is desired to include an antibacterial agent for application to the skin. Examples of such compositions include, but are not limited to, personal care compositions, antiperspirants, deodorants, body washes, shower gels, bar soaps, shampoo, hair conditioners, and cosmetics.

For antiperspirant/deodorant compositions, the carrier can be any carrier that is used for antiperspirants/deodorants. The carrier can be in the form of a stick, a gel, a roll-on, or an aerosol. For stick formulations, the carrier may include oils and/or silicones and gelling agents. An example of a formulation can be found in US2011/0076309A1, incorporated by reference herein.

Optional ingredients that can be included in an antiperspirant and/or deodorant formulation of the compositions of the invention include solvents; water-soluble alcohols such as C₂₋₈ alcohols including ethanol; glycols including propylene glycol, dipropylene glycol, tripropylene glycol and mixtures thereof; glycerides including mono-, di- and triglycerides; medium to long chain organic acids, alcohols and esters; surfactants including emulsifying and dispersing agents; amino acids including glycine; structurants including thickeners and gelling agents, for example polymers, silicates and silicon dioxide; emollients; fragrances; and colorants including dyes and pigments. If desired, an antiperspirant and/or deodorant agent additional to the antiperspirant active ingredient can be included, for example an odor reducing agent such as a sulfur precipitating agent, e.g., copper gluconate, zinc gluconate, zinc citrate, etc.

The composition can also optionally contain emollients in any desired amount to achieve a desired emollient effect. Emollients are known in the art and are used to impart a soothing effect on the skin. Non-volatile emollients are preferable. Classes of non-volatile emollients include non-silicone and silicone emollients. Non-volatile, non-silicone emollients include C₁₂₋₁₅ alkyl benzoate. The non-volatile silicone material can be a polyethersiloxane, polyalkyarylsiloxane or polyethersiloxane copolymer. An illustrative non-volatile silicone material is phenyl trimethicone. Non-limiting examples of emollients can be found in U.S. Pat. No. 6,007,799. Examples include, but are not limited to, PPG-14 butyl ether, PPG-3 myristyl ether, secondary alcohol ethoxylates (e.g. Tergitol sold by Dow Chemical Company, Midland, Mich.) stearyl alcohol, stearic acid and salts thereof, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, coconut oil, rice wax, shea butter, cocoa butter, glycyrrhizic acid, hydrocyethyl stearate amide.

The composition can contain a fragrance. Any known fragrance can be used in any desired amount. In one embodiment, the amount of fragrance is 0.01 to 10 wt. %.

Antioxidants may be added to the composition, preferably to act as ingredient protectants and for maintenance of long-term stability of the composition. Examples of antioxidants include, but are not limited to citric acid, butylated hydroxytoluene, pentaerythrityl tetra-di-t-butyl hydroxyhydrocinnamate.

The composition may also contain polymeric materials for thickening, such as polyamides, cellulose derivatives (e.g., hydroxypropylcellulose, hydroxypropyl methyl cellulose, etc.) and natural or synthetic gums, such as polyglycerides including agar, agarose, pectin, or guars or mixtures or combinations thereof. One class of materials worthy of attention for thickening a water-immiscible phase comprises derivatives of hydrolysed starch or other polysaccharides, including in particular esterified dextrins, such as dextrin palmitate. A further class of polymers that is particularly directed to structuring an oil phase containing a silicone oil comprises polysiloxane elastomers. Suspending agents such as silicas or clays such as bentonite, montmorillonite or hectorite, including those available under the trademark Bentone can also be employed to thicken liquid compositions according to the invention. The composition can be thickened with non-polymeric organic gellants, including selected dibenzylidene alditols (e.g. dibenzylidene sorbitol).

Any of the liquid antiperspirant/deodorant compositions can be applied to axillary areas to reduce sweat and/or odor. The compositions can be applied by hand or via their packaging.

The present invention moreover relates to a method for prophylaxis of skin irritation, a method for treatment of skin irritation, a method for reducing, eliminating or suppressing the irritating, preferably the skin-irritating, action of a substance or substance mixture, and a kit comprising (i) a formulation, a cosmetic product or a pharmaceutical product according to the present invention and, spatially separated, (ii) one or more substances or substance mixtures having an irritating, preferably a skin-irritating, action.

The antiperspirant compositions can be formulated into topical antiperspirant and/or deodorant formulations suitable for application to skin, illustratively a stick, a gel, a cream, a roll-on, a soft solid, a powder, a liquid, an emulsion, a suspension, a dispersion or a spray. The composition can comprise a single phase or can be a multi-phase system, for example a system comprising a polar phase and an oil phase, optionally in the form of a stable emulsion. The composition can be liquid, semi-solid or solid. The antiperspirant and/or deodorant formulation can be provided in any suitable container such as an aerosol can, tube or container with a porous cap, roll-on container, bottle, container with an open end, barrel, etc.

The compositions can be used in a method to reduce sweating by applying the composition to skin. In certain embodiments, the application is to axilla. Also, the compositions can be used to kill bacteria by bringing the bacteria into contact with the composition.

Thus the invention provides (i) a method for controlling perspiration comprising applying to skin an antiperspirant effective amount of a formulation of any embodiment embraced or specifically described herein, e.g., any of Compositions 1 et seq.; and (ii) a method for controlling odor from perspiration comprises applying to skin a deodorant effective amount of a formulation of any embodiment embraced or specifically described herein, e.g., any of Compositions 1 et seq.

In this text, the term “skin” also includes the “mucous membrane” (mucosa), especially the mucous membrane of mouth, throat, gums, nose, respiratory and gastrointestinal tract (“GI tract”). In the cosmetics and pharmaceuticals industry, there is a constant need for agents having an irritation-reducing action.

The mucous membranes, which line various body cavities that are exposed to the external environment and internal organs (e.g. mouth and throat), and the skin in general (in particular the epidermis) are—as barrier organs of the human organism—subjected to external influences to a particular extent. Many intrinsic (e.g. genetic predisposition) and extrinsic (e.g. damage to the skin barrier, action of UV light, irritating or allergy-inducing substances) factors can lead to skin irritation. In connection with this application, “skin irritation” is to be understood as meaning any change to the skin which induces sensorial malaise in humans or animals and/or is characterized by dry, reddened and/or inflamed skin symptoms. The term “sensorial malaise” here of course also includes states such as itching or pain. Skin irritation can include, in particular, several different skin states such as: delicate skin, sensitive skin, including sensitive scalp, easily injured skin, atopic skin (atopy), irritated skin or inflamed skin, which may manifest itself in a reddening of the skin, the so-called erythema. Skin irritations can further include irritations of the oral cavity, like periodontitis, gingivitis and the like, as described in more detail below, irritations like rhinosinusitis (common cold), sinusitis, pharyngitis/tonsillitis and the like, as described in more detail below and in US 2009/0238905, incorporated herein by reference, and irritations of the gastrointestinal tract, as described in more detail below and in US 2009/0238905, incorporated herein by reference.

The compositions and formulations as provided herein are described and claimed with reference to their ingredients, as is usual in the art. As would be evident to one skilled in the art, the ingredients may in some instances react with one another, so that the true composition of the final formulation may not correspond exactly to the ingredients listed. Thus, it should be understood that the invention extends to the product of the combination of the listed ingredients.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Unless otherwise specifically identified, the ingredients for use in the compositions and formulations of the present invention are preferably cosmetically acceptable ingredients. By “cosmetically acceptable” is meant suitable for use in a formulation for topical application to human skin. A cosmetically acceptable excipient, for example, is an excipient which is suitable for external application in the amounts and concentrations contemplated in the formulations of this invention, and includes for example excipients which are “Generally Recognized as Safe” (GRAS) by the United States Food and Drug Administration.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

### Roll-On Antiperspirant Compositions

Compositions for roll-on forms of an antiperspirant containing CBD oil are described in Tables 1 and 2:

TABLE 1
Roll-on Composition 1

| Description | wt. % |
|---|---|
| Water | 40-60 |
| Hydroxypropyl methylcellulose | 0.5-1.5 |
| Propylene Glycol | 5-15 |
| Polyethylene Glycol (PEG 600) | 1-3 |
| 50% Aluminum Chlorhydroxide Soln. | 20-40 |
| Tergitol 15-S-12 | 1.5-2.5 |
| Fragrance agents | 0.5-1.5 |
| Cannabis Sativa Seed Oil (5% CBD) | 0.001-5 |
| Total components | 100.00 |

TABLE 2
Roll-on Composition 2

| Description | wt. % |
|---|---|
| Water | 50-70 |
| Steareth-20 | 0.5-1.5 |
| Caprylyl Glycol | 0.01-0.5 |
| Stearyl Ether | 1-2 |
| Steareth-2 | 1.5-3 |
| Soybean oil | 2-4 |
| Butylated hydroxytoluene | 0.01-0.1 |
| EDTA 62% Soln. | 0.01-0.5 |
| 50% Aluminum Chlorhydroxide Soln. | 20-40 |
| Fragrance agents | 0.5-1.5 |
| Cannabis Sativa Seed Oil (5% CBD) | 0.001-5 |
| Total Components | 100.00 |

### Example 2

### Aerosol Antiperspirant Compositions

Compositions for aerosol forms of an antiperspirant containing CBD oil are described in Tables 3 and 4:

TABLE 3
Aerosol Composition 1

| Material Description | wt. % |
|---|---|
| Isopropyl Palmitate | 15-25 |
| Isopropyl myristate | 15-25 |
| C₁₂₋₁₅ Alkyl Benzoate | 5-18 |
| Soybean oil | 2-6 |
| Bentone | 1.5-3 |
| Propylene Carbonate | 0.1-1 |
| Aluminum Chlorhydroxide Powder | 30-50 |
| Cannabis Sativa Seed Oil (5% CBD) | 0.001-5 |
| Total Components | 100.00 |

TABLE 4
Aerosol Composition 2

| Material Description | wt. % |
|---|---|
| 94% Ethyl Alcohol Soln. | 90-98 |
| Fragrance | 1-2 |
| Farnesol | 0.01-0.2 |
| Cannabis Sativa Seed Oil (5% CBD) | 0.001-5 |
| Total Components | 100.00 |

### Example 3

### Solid Antiperspirant Compositions

Compositions for solid forms (i.e., antiperspirant sticks) of an antiperspirant containing CBD oil are described in Tables 5 and 6:

TABLE 5
Antiperspirant Stick Composition 1

| Description | wt. % |
|---|---|
| Activated Aluminum Zirconium | 8-18 |
| Tetrahydroclorex Glycine | |
| Palm Kernel Oil | 30-45 |
| Cyclomethicone | 5-15 |
| C₁₂₋₁₅ Alkyl Benzoate | 10-25 |
| PEG-8 Distearate | 2-6 |
| Soybean Oil | 2-6 |
| 50% Citric Acid Soln. | 0.01-0.1 |
| Pentaerythrityl tetra-di-t-butyl | 0.001-0.1 |
| hydroxyhydrocinnamate | |
| Butylated hydroxytoluene | 0.01-1 |
| Synthetic Wax | 5-15 |
| Cannabis Sativa Seed Oil (5% CBD) | 0.001-5 |
| Total Components | 100.00 |

TABLE 6
Antiperspirant Stick Composition 2

| Description | wt. % |
|---|---|
| Polypropylene Glycol | 55-75 |
| Sodium Stearate | 5-15 |
| Stearyl Alcohol | 0.01-1 |
| Water | 15-25 |
| EDTA 62% Soln. | 0.001-0.01 |
| Sodium Chloride | 0.1-1 |
| Colorants | 0.0001-0.001 |
| Fragrance | 1-3 |
| Cannabis Sativa Seed Oil (5% CBD) | 0.001-5 |
| Total Components | 100 |

### Example 4

### Comparison of Various Solid Antiperspirant Compositions

Antiperspirant compositions were prepared according to the following in Tables 7, 8 and 9 and were compared for appearance, color and odor.

TABLE 7
Solid Composition 1

| Description | wt. % |
|---|---|
| Palm Kernel Oil | 30-40 |
| Dicaprylyl Ether | 5-15 |
| Soybean oil | 4-8 |
| Castor oil | 4-8 |
| Stearyl Alcohol | 9-18 |
| Aluminum Chlorhydroxide Powder | 25-35 |
| Olea Europaea Leaf Extract | 0.001-0.5 |
| Maltodextrin | 0.001-0.5 |
| Silica | 0.001-0.5 |
| Fragrance | 0.1-2 |
| Cannabis Sativa Seed Oil (5% CBD) | 0.001-5 |
| Total Components | 100.00 |

TABLE 8
Solid Composition 2

| Description | wt. % |
|---|---|
| Palm Kernel Oil | 30-45 |
| Stearyl Alcohol | 15-25 |
| Caprylic/Capric Triglyceride | 5-10 |
| White Beeswax | 1-5 |
| Dicaprylyl Ether | 5-15 |
| Soybean oil | 6-10 |
| Castor oil | 6-10 |
| Zinc Oxide | 1-3 |
| Cannabis Sativa Seed Oil (5% CBD) | 0.001-5 |
| Fragrance | 1-2 |
| Total Components | 100.00 |

TABLE 9
Solid Composition 3

| Description | wt. % |
|---|---|
| Propylene Glycol | 55-75 |
| Water | 20-30 |
| Sodium Stearate | 4-8 |
| Glyceryl Monolaurate | 0.1-2 |
| Ascorbic Acid | 0.01-0.1 |
| Aloe | 0.01-0.1 |
| Cannabis Sativa Seed Oil (5% CBD) | 0.001-5 |
| Total Components | 100.00 |

The compositions were compared to a Control Composition for characteristics in terms of compression, color, odor and general appearance. The tested compositions showed satisfactory characteristics. The results are summarized in Table 10 below:

TABLE 10

| | Com- | | | |
|---|---|---|---|---|
| | pression | | | |
| Formula | (g) | Color | Odor | Appearance |
| Control | 2000-5000 | White | Match | Solid white |
| Formulation | | | to Std | opaque |
| Solid Com- | 3100 | White | Match | Solid white |
| position 2 | | | to Std | opaque |

Control Formulation

| Description | wt. % |
|---|---|
| Palm Kernel Oil | 39.65 |
| Stearyl Alcohol | 18.38 |
| Caprylic/Capric Triglyceride | 7.17 |
| White Beeswax | 3.46 |
| Dicaprylyl Ether | 10.21 |
| Soybean oil | 8.17 |
| Castor oil | 8.68 |
| Zinc Oxide | 2.00 |
| Cannabis Sativa Seed Oil (5% CBD) | 0.001-5 |
| Fragrance | 0.30 |
| Total Components | 100.00 |

### Example 5

### Effect of CBD and Hemp Seed Oil Compositions on Various Inflammatory Biomarkers

Tests were carried out to analyze the effect of CBD on the anti-inflammatory and anti-irritation effects of CBD present in varying amounts in antiperspirant compositions. The base composition used for the testing did not contain any cannabidiol (CBD). Further Formulations containing CBD were prepared by adding varying amounts of cannabis sativa seed oil to the Base Formulation. Formulations 1 and 2 were prepared with cannabis sativa seed oil containing 1% CBD, and Formulations 3 and 4 were prepared with cannabis sativa seed oil containing 5% CBD. The test formulations were therefore prepared as follows:

TABLE 12
Test Formulations

| | Description | wt. % |
|---|---|---|
| Formulation 1 | Cannabis sativa seed oil | 0.5 |
| | (1% CBD*) | |
| Formulation 2 | Cannabis sativa seed oil | 1.0 |
| | (1% CBD*) | |
| Formulation 3 | Cannabis sativa seed oil | 0.5 |
| | (5% CBD*) | |
| Formulation 4 | Cannabis sativa seed oil | 1.0 |
| | (5% CBD*) | |

*Wherein the amount (wt %) of CBD is relative to the weight of the cannabis sativa seed oil

These compositions are slurried and applied to an assay in order to test their effect on modulating various inflammatory and irritation biomarkers. Specifically, the effects were observed for IL-1α, IL-6, IL-8, Natural Moisturizing Factor (NMF) expressed via Caspase 14, Filaggrin, as well as the transepithelial electrical resistance (TEER) of the samples. IL-1α, IL-6, and IL-8 are known pro-inflammatory cytokines. NMF plays a role in maintaining adequate skin hydration by maintaining plasticity of the skin; allowing hydrolytic enzymes to function in the process of desquamation; and contributing to optimum stratum corneum barrier function. Filaggrin is a filament-associated and cross-linked protein that contributes to the mechanical strength of the stratum corneum, or the uppermost layer of the epidermis. Transepithelial/transendothelial electrical resistance (TEER) is a widely accepted quantitative technique to measure the integrity of tight junction dynamics in cell culture models of endothelial and epithelial monolayers. It is believed that a beneficial antiperspirant would contribute to a reduction in inflammation/irritation and an increase in the hygroscopicity and strength of the skin. Specifically, it is believed that a beneficial antiperspirant would cause a reduction in inflammatory cytokines (e.g., IL-1α, IL-6, IL-8), and an increase in observed NMF, Filaggrin, as well as the measured value for TEER. The observed results are summarized below.

TABLE 13
Change in IL-1α Levels

| Sample | IL-1α Levels |
|---|---|
| Base Formulation | 91.4 |
| Formulation 1 | 26.5 |
| Formulation 2 | 74.4 |
| Formulation 3 | 32 |
| Formulation 4 | 49.4 |

TABLE 14
Change in IL-6 Levels

| Sample | IL-6 Levels |
|---|---|
| Base Formulation | 0.76 |
| Formulation 1 | 0.13 |
| Formulation 2 | 0.49 |
| Formulation 3 | 0.49 |
| Formulation 4 | 1.3 |

TABLE 15
Change in IL-8 Levels

| Sample | IL-8 Levels |
|---|---|
| Base Formulation | 67.9 |
| Formulation 1 | 65.3 |
| Formulation 2 | 74.5 |
| Formulation 3 | 60 |
| Formulation 4 | 79.2 |

TABLE 16
Change in Caspase 14 Levels

| Sample | Caspase 14 Levels |
|---|---|
| Base Formulation | 11.4 |
| Formulation 1 | 11.3 |
| Formulation 2 | 11.5 |
| Formulation 3 | 10.7 |
| Formulation 4 | 12.2 |

TABLE 17
Change in Filaggrin Levels

| Sample | Caspase 14 Levels (pg/mL) |
|---|---|
| Base Formulation | 603.9 |
| Formulation 1 | 338 |
| Formulation 2 | 543.3 |
| Formulation 3 | 333.9 |
| Formulation 4 | 344 |

TABLE 18
Observed TEER

| Sample | TEER |
|---|---|
| Base Formulation | 850 |
| Formulation 1 | 1615 |
| Formulation 2 | 805 |
| Formulation 3 | 1714 |
| Formulation 4 | 1142 |

As shown in Tables 13-15, the tested Formulations generally performed better than the Base Formulation in reducing the expression of the inflammatory biomarkers IL-1α, IL-6, and IL-8. In particular, each of Formulations 1-4 resulted in a decrease in IL-1α in comparison with the Base Formulation, each of Formulations 1-3 resulted in a decrease in IL-6, and Formulations 1 and 3 resulted in a decrease of IL-8. Surprisingly, the compositions containing 0.5 wt. % of cannabis sativa seed oil (i.e., Formulations 1 and 3) performed generally better than the compositions containing 1.0 wt. % cannabis sativa seed oil (i.e., Formulations 2 and 4), which is a surprising outcome. Similarly, the compositions containing 0.5 wt. % of cannabis sativa seed oil showed an overall increase in the strength of the skin tissue via increases in Caspase 14 expression. TEER measurements showed significant increases following the application of both Formulation 1 and 3.

### Example 6

In vitro studies were conducted on MatTek human reconstructed tissue model. IL-1α protein released in culture media was quantified with an Elisa kit. IL-1α, IVL, FLG and LOR genes were quantified with gene expression study.

Tissues were normalized in 6-well plates with 1.2 ml media/well for overnight incubation (5% CO2% 37° C.). The following day, the tissue samples were transferred to new set 6-well plates with fresh media (1.5 ml). 30 μl of 0.1% SLS was added to each tissue sample and all samples were incubated for 1 hour. The samples were then washed 8 times using PBS and moved to fresh media. The test deodorant composition in stick form was applied to the samples with a paint brush. The brush was saturated with the deodorant and was applied to tissue three to each tissue sample. The samples were incubated (5% CO2% 37° C.) for 24 hours. On the following day all samples were collected for testing.

IL-1α was quantified with the Elisa kit. Afterward, RNA was extracted from tissue and gene expression was quantified for Ceramide synthase 3 (CersS3), Involucrin (IVL), Filaggrin (FLG) and Loricrin (LOR). GAPDH and PPIA were used as endogenous controls. After calculating Rq values, they were converted to percentages (%) and expressed as % of change relative to control group.

The test deodorant compositions were prepared as disclosed in Example 5 above, and the Control Formulation was prepared as disclosed in Example 4. The compositions were additionally compared to a Commercial Comparator that did not contain any CBD.

The expression of IL-1α and the expression of the selected genes are summarized in Table 19 below.

TABLE 19

| Product | | | | | |
|---|---|---|---|---|---|
| Commercial | IL-1 a | CERS3 | FLG | IVL | LOR |
| Comparator | 717.2 | 1.03 | −13.8 | 46.4 | 34.8 |
| Control | | | | | |
| Formulation | 0 | 0 | 0 | 0 | 0 |
| Formulation 1 | 21.4 | 4.9 | −1.9 | 8 | 2.2 |
| Formulation 2 | −22.3 | 25.7 | −1 | −2.4 | 1.3 |
| Formulation 3 | −12.2 | 33.4 | 52.6 | 6.4 | 44.7 |
| Formulation 4 | −9.2 | 60.6 | 78.2 | 61 | 81.7 |

As shown above, the test compositions containing with CBD demonstrated skin benefits like anti-irritation not only at the gene level but also at protein level. The gene expression study also showed the benefits of enhancing skin barrier function.

While the present invention has been described with reference to embodiments, it will be understood by those skilled in the art that various modifications and variations may be made therein without departing from the scope of the present invention as defined by the appended claims.

1. A personal care composition for application to the skin or hair comprising a cosmetically acceptable carrier and about 0.1 to about 5.0 wt. % of a cannabinoid source, based on the total weight of the composition.

2. A composition according to claim 1, wherein the cannabinoid source comprises Cannabis sativa seed oil or Hemp seed oil.

3. A composition according to claim 1, wherein the cannabinoid source comprises Cannabis sativa seed oil.

4. A composition according to claim 1, wherein the cannabinoid comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), and combinations thereof.

5. A composition according to claim 1, wherein the cannabinoid is a non-psychoactive cannabinoid.

6. A composition according to claim 1, wherein the composition is substantially free of Δ9-tetrahydrocannabinol (THC).

7. A composition according to claim 1, wherein the cannabinoid source comprises a cannabinoid selected from cannabichromene (CBC), cannabigerol (CBG), cannabidiol (CBD), cannabinol (CBN), and combinations thereof.

8. A composition according to claim 1, wherein the cannabinoid source comprises cannabidiol (CBD).

9. A composition according to claim 8, wherein the CBD is present in an amount of 0.005 wt. % to 3.0 wt. %, 0.01 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.

10. A composition according to claim 1, further comprising a metal-containing antiperspirant active ingredient.

11. A composition according to claim 1 in a cosmetically acceptable base suitable for application to the skin, the base comprising one or more of water-soluble alcohols (such as C₂₋₈ alcohols including ethanol); glycols (including propylene glycol, dipropylene glycol, tripropylene glycol and mixtures thereof); glycerides (including mono-, di- and triglycerides); medium to long chain organic acids, alcohols and esters; surfactants (including emulsifying and dispersing agents); additional amino acids; structurants (including thickeners and gelling agents, for example polymers, silicates and silicon dioxide); emollients; fragrances; and colorants (including dyes and pigments).

12. A composition according to claim 1, further comprising a soothing agent.

13. A composition according to claim 1, wherein the composition is an antiperspirant and/or deodorant, e.g., an antiperspirant stick, an aerosol antiperspirant spray, or a liquid roll-on antiperspirant, cream antiperspirant.

14. A method of reducing perspiration, reducing body odor, or killing bacteria comprising applying an antiperspirant effective amount of a composition according to claim 1 to the skin.

15. A stick deodorant or antiperspirant composition for application to the skin comprising a cosmetically acceptable carrier and about 0.001 to about 5.0 wt. % of a cannabinoid source, based on the total weight of the composition.

16. A roll-on deodorant or antiperspirant composition for application to the skin comprising a cosmetically acceptable carrier and about 0.001 to about 5.0 wt. % of a cannabinoid source, based on the total weight of the composition.

17. An aerosol deodorant or antiperspirant composition for application to the skin comprising a cosmetically acceptable carrier and about 0.001 to about 5.0 wt. % of a cannabinoid source, based on the total weight of the composition.

18. A solid deodorant or antiperspirant composition for application to the skin comprising a cosmetically acceptable carrier and about 0.001 to about 5.0 wt. % of a cannabinoid source, based on the total weight of the composition.
